(19) United States
(12) Patent Application Publication
(10) Pub. No.: US 2009/0069611 A1
(43) Pub. Date: Mar. 12, 2009

(54) Stoichiometric or cyclical re-hydrogenation of silicon, nanodiamond, or nanocarbon surfaces using hydrocarbons as sources of hydrogen

(76) Inventor: Charles Martin Lukehart, Nashville, TN (US)

Correspondence Address: JOHN BRUCKNER, P.C., P.O. BOX 490, FLAGSTAFF, AZ, 86002, US

(21) Appl. No.: 12/001,077

(22) Filed: Dec. 6, 2007

Related U.S. Application Data
(60) Provisional application No. 60/873,001, filed on Dec. 6, 2006.

(51) Int. Cl. C07C 5/00

(52) U.S. Cl. 585/250

(57) ABSTRACT

Methods are described for stoichiometric or cyclical re-hydrogenation of silicon, nanodiamond, or nanocarbon surfaces using hydrocarbons as sources of hydrogen. A method includes forming reactive sites on an adsorbate-substrate by non-thermal, non-electronic resonant photodesorption of a gas from the adsorbate-substrate; reacting the reactive sites with a functional radical; and cyclically repeating the steps of forming and reacting. The gas includes hydrogen and reacting includes re-hydrogenation of the reactive sites, the functional radical includes a hydrocarbon, the adsorbate-substrate is selected from silicon, nanodiamond or nanocarbon and resonant photodesorption includes a vibrational stretch mode.

### CROSS-REFERENCE TO RELATED APPLICATION

This application claims a benefit of priority under 35 U.S.C. 119(e) from copending provisional patent application U.S. Ser. No. 60/873,001, filed Dec. 6, 2006, the entire contents of which are hereby expressly incorporated herein by reference for all purposes.

### FIELD OF THE INVENTION

The present invention relates to the extraction of hydrogen from hydrocarbons via desorption of hydrogen from adsorbate-substrate systems and the utilization of the desorbed hydrogen and hydrogen-free substrates in multiple applications.

### BACKGROUND OF THE INVENTION

The efficient desorption of hydrogen from a wide variety of substrates without damage to either the desorbed hydrogen or substrate has long been the aim of past research. For example, microelectronic devices are conventionally built from multiple layers of silicon. In order to keep silicon surfaces from oxidizing before construction of the devices in which they are incorporated, semiconductor manufactures routinely “passivate” silicon surfaces by exposing them to hydrogen atoms that attach to all the available silicon bonds. However, this requires the removal of the hydrogen atoms before new layers of silicon can be added during device manufacture. “Desorbing” the hydrogen thermally, the current method for doing so, requires high temperatures and adds substantially to the production cost. In addition, the high temperatures create thermal defects in the chips, thereby reducing chip yields. It is also desirable to remove hydrogen atoms from a variety of other substrates such as nanocrystal diamonds and the like.

Since the invention of the laser, chemists have been trying to use it to drive chemical reactions along non-thermal pathways. However, when a molecule is heated, the weakest bond is the first to break. Attempts to tune lasers to selectively break bonds have been thwarted by the rapidity with which irradiated molecules convert the laser light energy into thermal energy, thereby resulting in the destruction of other than the targeted bonds.

Photon stimulated desorption is a powerful tool to study fundamental processes in adsorbate surface systems, as well as to achieve selective surface reactions for controlled surface processing. Photons are easily directed and tuned in energy to induce transitions in atomic and molecular states with high spatial and temporal precision. Direct adsorbate-surface bond breaking by electronic excitation using ultraviolet light has been reported. However, visible and infrared (1R) stimulated desorption processes studied so far generally involve indirect mechanisms, such as light-induced substrate heating and, in physisorbed systems, energy transfer from internal molecular excitation to molecular translational motion away from the surface. Selective bond scission at these lower energies is desirable, but has proven challenging because of rapid energy delocalization from the mode of excitation.

### SUMMARY OF THE INVENTION

One embodiment of the present invention relates to a method for the photodesorption of hydrogen from a hydrogen-adsorbate substrate comprising subjecting the substrate to laser radiation tuned to a photon energy resonant with the hydrogen-substrate vibrational stretch mode for a time sufficient to result in the non-thermal, non-electronic desorption of at least some of the hydrogen from the substrate.

Another embodiment of the invention concerns a method of creating reactive sites on a substrate containing adsorbed hydrogen comprising photodesorbing hydrogen therefrom by subjecting the substrate to laser radiation tuned to a photon energy resonant with the hydrogen-substrate vibrational stretch mode for a time sufficient to result in the non-thermal, non-electronic desorption of at least some of the hydrogen from the substrate, thereby creating reactive sites thereon, wherein the reactive sites are capable of reacting with chemically reactive radicals.

Other embodiments of the invention relate to the hydrogen-desorbed substrates as well as applications of the desorbed hydrogen and thus activated substrates for a variety of purposes.

### BRIEF DESCRIPTION OF THE DRAWINGS

The drawings accompanying and forming part of this specification are included to depict certain embodiments of the invention. A clearer concept of embodiments of the invention, and of components combinable with embodiments of the invention, and operation of systems provided with embodiments of the invention, will be readily apparent by referring to the exemplary, and therefore nonlimiting, embodiments illustrated in the drawings. Embodiments of the invention may be better understood by reference to one or more of these drawings in combination with the following description presented herein. It should be noted that the features illustrated in the drawings are not necessarily drawn to scale.

FIGS. 1A-1B illustrate (1A) wavelength dependence of hydrogen desorption yield; and (1B) polarization dependence of hydrogen desorption yield.

FIGS. 2A-2B illustrate (2A) deconvolution of the FTIR absorption spectrum within the C—H stretching band on an intrinsic CVD polycrystalline diamond film; and (2B) preliminary data of wavelength dependence of H desorption from a polycrystalline diamond film where the broad peak is due to contributions from the C—H bonds on various diamond surfaces.

FIGS. 3A-3B illustrate (3A) hydrogen quadratic power dependence; and (3B) hydrogen polarization dependence on an intrinsic CVD polycrystalline diamond film.

FIGS. 4A-4B illustrate (4A) hydrogen on Si(111) wavelength dependence; and (4B) fluence/intensity dependence.

FIGS. 5A-5C illustrate (5A) FTIR absorption spectra; and (5B-5C) wavelength dependence of CVD intrinsic polycrystalline diamond film.

FIG. 6 illustrates a light-induced catalytic cycle for continuous production of H₂ from hydrocarbons.

### DETAILED DESCRIPTION OF THE INVENTION

The present invention is predicated on the discovery that the hydrogen/adsorbate-substrate bond may be selectively and non-thermally broken to produce desorbed hydrogen and a substrate reactively activated at the hydrogen-desorption sites, utilizing laser radiation tuned to a photon energy resonant with the hydrogen-substrate vibrational stretch mode. It is an unexpected advantage of the method of the invention that the hydrogen is desorbed in pairs as H₂ rather than as individual H atoms.

The laser desorption method of the invention:

1) generates surprisingly little heat. For example, in the infrared wavelengths typically used on hydrogen/silicon adsorbate systems silicon is basically transparent to the infrared radiation. Therefore, photons that don't contribute to the desorption process are either reflected by the surface or pass through it, thereby generating little or no heat. Further evidence of this fact is that, in the experiments with hydrogen-deuterium mixtures, less than 5 percent of the desorbed material was in the form of hydrogen-deuterium or deuterium-deuterium pairs, indicating that very little of the laser energy involved in desorbing the hydrogen is converted to heat.

2) Exhibits a high degree of selectivity. With the hydrogen/deuterium mixture, it has been demonstrated that large numbers of hydrogen atoms could be detached/desorbed without detaching many of the deuterium atoms. This result indicates that hydrogen can also selectively be removed from certain surface locations, but not others. For example, the strength of the bonds holding hydrogen atoms to the silicon atoms located on stairstep edges are stronger than those on flat areas. Thus, the laser can be tuned with enough precision to detach just the stair-step hydrogen atoms.

This kind of selectivity provides a way to control the growth of nanoscale structures with an unprecedented degree of precision. By selectively removing the hydrogen atoms from the ends of nanowires, e.g., one can control and direct their growth. Currently, nanowire growth is a random process. Another possibility is encapsulation. For example, fill the etched cavity in a silicon wafer with a material and then use the laser to selectively desorb the hydrogen atoms from the lip of the cavity. If this is done in the presence of silicon vapor then one can grow an impermeable lid of silicon over the cavity. Yet another application is the construction of quantum computers. Indeed, once hydrogen has been desorbed from the substrate, substances like silanes and hydrocarbons such as methane can be disassociated over the resulting vacant sites to grow silicon or carbon, respectively.

Normally one can only grow silicon at 600 C. The method of the invention will enable high quality growth and growth on specific facets at much lower temperatures, perhaps as low as room temperature. If one has a sensor or an integrated circuit, this method would enable one to add silicon without disturbing existing electronics (abrupt interfaces and wires) since it occurs at such low temperatures. It could also enable bonding of silicon wafers for encapsulation or for growth of specific structures designed for encapsulation. It could be used to make nanowires directed towards contacts. The list of potential applications is virtually endless. Nanowire devices of Si or diamond could be grown, silicon or diamond could be grown between room temperature and several hundred degrees Celsius. Silicon or diamond could be grown between room temperature and several hundred degrees Celsius, without disturbing interconnects or causing thermal diffusion of existing materials in an integrated circuit. Hermetic seals could be formed either by extending a silicon feature or by bonding two silicon wafers.

As noted above, a key step in the manufacture of computer chips is the desorption of hydrogen from passivated silicon substrates. The method of the invention enables the highly cost efficient desorption of hydrogen from silicon to produce heretofore unobtainable high-quality silicon chips. For example, one application is the use of this technique to manufacture field effect transistors (FETs) that operate at speeds about 40 percent faster than ordinary transistors. It should be possible to reduce the processing temperature of manufacturing FETs by 100 degrees Celsius which will dramatically improve yields.

When the laser is scanned through the frequencies calculated to resonate with, e.g., the silicon-hydrogen bond, it was found that the rate of hydrogen desorption peaked at 4.8 microns. Next, the laser beam was polarized so that the electrical field in the photons pointed in the same direction as the silicon-hydrogen bonds, thereby producing very selective hydrogen desorption results.

In the non-limiting examples set forth below, the resonant photodesorption of hydrogen from a Si(111) surface using tunable infrared radiation is reported. The wavelength dependence of the desorption yield peaks at 0.26 e V, the energy of the Si—H vibrational stretch mode. The desorption yield is quadratic in the infrared intensity. A strong HID isotope effect rules out thermal desorption mechanisms, and electronic effects are not applicable in this low energy regime. It is shown that the process is resonant with the Si—H vibrational energy and displays an unusual and surprising dependence on excitation intensity, which cannot be explained by either thermal or electronic processes. Successful elucidation of this fundamental excitation mechanism constitutes a major advance in surface science, and its implementation enables site selective desorption at low temperatures.

The H/Si(111) structure is a well-characterized adsorbate system, ideal for the study of fundamental surface processes. The Si—H bond is perpendicular to the Si(111) surface, with a bond energy estimated between 3.15 eV and 3.35 eV, and a vibrational stretch energy of 0.26 e V at the terrace sites. In experiments, the Si—H vibrational stretch mode is resonantly excited by IR photons. The Si substrate is transparent to mid-IR illumination, minimizing electronic excitation and direct laser-induced heating. The desorption process has important connections to technology on the hydrogen-desorbed (e.g., Si surface). Because hydrogen desorption is an important component of Si chemical vapor deposition, modifying the H desorption mechanism will have a large impact on growth. In particular, the vibrational energy of an Si—H bond at the terrace site of the Si (111) surface differs from that at the step site by 51.8 cm⁻¹, enabling site-selective desorption of H adatoms by IR irradiation. Such a photolytic process can efficiently treat a large area, and modify the type of sites available for epitaxial growth. This can be compared to nanoscale lithography of H on Si achieved using the scanning tunneling microscope (STM).

The experiment was performed in an ultra-high vacuum (UHV) system at room temperature. The base pressure was ˜3.0×10−¹⁰ Torr. The sample was prepared in air using HF etching to protect the Si surface from oxidation and contamination, and then cleaned by direct current heating in the UHV chamber. The clean surface was verified by the strong Si(111)-7×7 low energy electron diffraction (LEED) pattern. Ultra-high purity H₂ gas was introduced into the UHV chamber. A tungsten filament at 2000 K was used to dissociate H₂ molecules, thus efficiently forming the Si—H bonds at the Si(111) surface. After a hydrogen dose of 3×10⁻⁶ Torr for 120 seconds, an area of 5 mm by 4 mm Si surface was uniformly covered with hydrogen atoms, as evidenced by the Si(111)-1×1 LEED pattern and the uniform desorption behavior at different spots when all other desorption conditions were kept the same. Thermal desorption spectra showed only one peak at ˜820 K, indicating that H was adsorbed exclusively as a monohydride phase on the Si surface.

The free-electron laser utilized provides a maximum of 100 mJ per macropulse at a repetition rate of 30H₂. Each macropulse is composed of ˜10⁴ micropulses of 1 ps duration, spaced 350 ps apart. The spectral width of the PEL emission is ˜50-90 cm⁻¹. The IR radiation was guided into the chamber through a CaF₂ viewport and focused onto the sample with a spot size of 0.8 mm. Its power and polarization could be adjusted with an attenuator and polarizer outside the chamber. The sample surface was positioned to make a 30° angle with the linearly polarized beam. The total electric field above the Si surface is the sum of the incident and the reflected fields. For the Si substrate with refractive index 3.42 and this incident angle of 60°, the direction of the total electric field above the Si surface is close to the surface normal, namely the direction of the Si—H bond. The field projection in this direction is (1+rₚ)E cos θ cos 30°, with E the incident field, rₚ the reflection coefficient of the p-polarized component, and θ the angle between the FEL electric field and the incident plane. The effective PEL fluence in the direction of the Si—H bond can be varied by changing either the incident intensity E² or the polarization angle θ.

The sample was exposed to the PEL illumination at room temperature, and hydrogen consequently desorbed was pumped away by the ion pump at the same time. An infrared pyrometer focused on the sample detected no temperature rise during desorption, showing no FEL-induced heating of the bulk sample, though not ruling out a local transient temperature increase. A quadruple mass analyzer was employed to record the H₂ partial pressure as a function of time once the PEL shutter was open. After desorption, an experimental parameter, such as the PEL wavelength or polarization, was varied, and the PEL focus was moved to a new sample position that had not been exposed to the PEL illumination. When the PEL beam was not incident on the Si surface, no desorption signal was measured, indicating that there was no contribution from scattered PEL radiation.

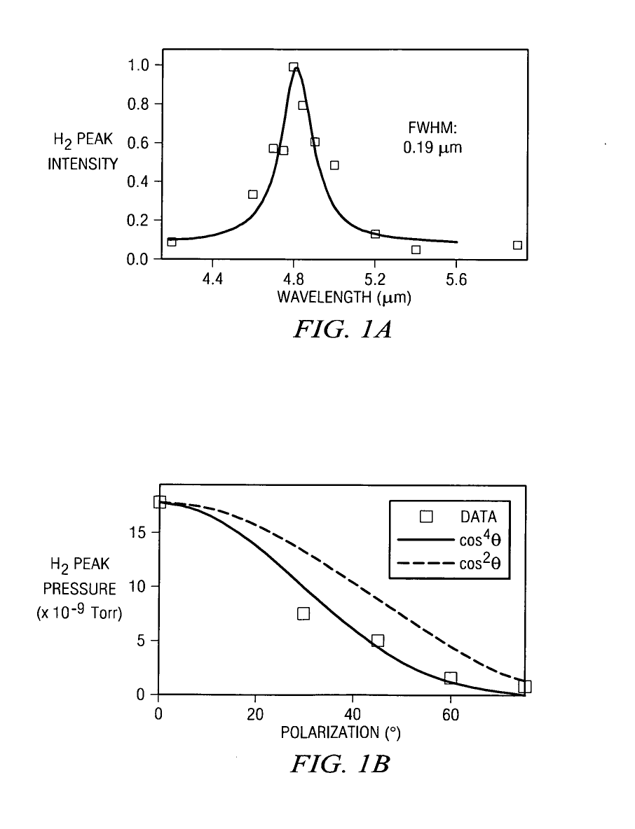

To study the wavelength dependence of H₂ photodesorption, the H₂ partial pressure was monitored as a function of time over a range of PEL wavelengths with the fluence kept fixed and the polarization set to θ=0° (FIG. 1A). The signal quickly rose to its peak, then decayed exponentially with a rate in agreement with the pumping speed of the vacuum system. The fine structure of the desorption curves (the inset of FIG. 1A) features step-like increases. The interval of 33 ms is in good agreement with the 30H₂ repetition rate of PEL macropulse, indicating that these increases resulted from desorption by each PEL macropulse. The H₂ partial pressure is a measure of the total number of H₂ molecules in the UHV chamber. Therefore the magnitude of measure of the total number of H₂ molecules in the UHV chamber. Therefore the magnitude of each jump is taken as a measure of the number of H₂ molecules desorbed by each macropulse, i.e., the desorption yield.

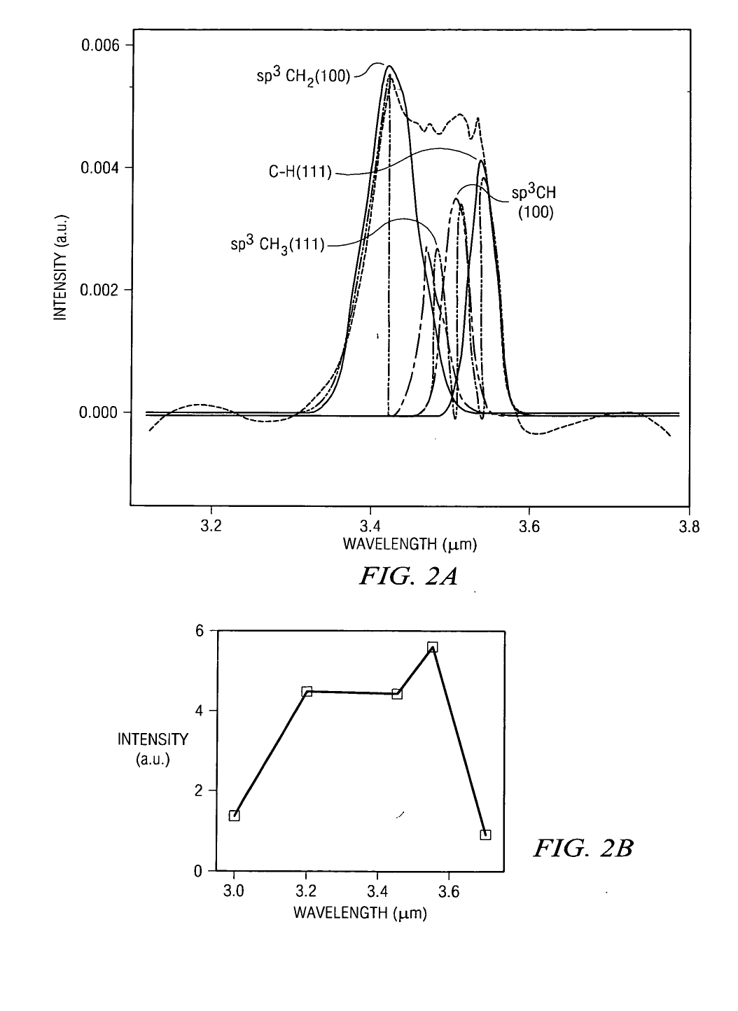

The desorption yield peaked at a wavelength of 4.8 μm (FIG. 1B), corresponding to 0.26 eV, the energy of the vibrational stretch mode of the Si—H bond at the terrace sites of the Si(111) surface. A Lorentzian fit yielded a full width at half maximum (FWHM) of 0.19 μm (82.5 cm⁻¹), close to the FEL linewidth, which is the limiting factor in resolving wavelength. Because the vibrational energy of the Si—H bond at the terrace site differs from that at the step site by 51.8 cm⁻¹, this resonance effect suggests that wavelength selective site desorption would be readily possible with an incident beam of appropriately narrow linewidth. In addition, desorption was unobservable when the wavelength was far from resonance, ruling out a desorption mechanism due to simple, direct laser heating. A study of the polarization dependence of the desorption yield provides further insight into the desorption mechanism. For bulk-like thermal processes, possibly due to impurities or defects, the desorption yield is not expected to be very sensitive to the polarization angle. For direct bond-breaking processes via electronic excitation, the yield should be a simple quadratic function of the PEL electric field projected in the direction of Si—H bond, i.e., a linear dependence on the incident intensity, and follow a cos² θ curve as illustrated (FIG. 2A). In the experiment with constant PEL fluence, however, the desorption yield scales with the incident polarization as cos⁴ θ (FIG. 2A). This behavior indicates that the observed desorption does not result from a thermal process caused by direct laser heating, but from the direct interaction between the incident field and the Si—H bonds. Furthermore, the cos⁴ θ dependence implies a quadratic dependence on the incident intensity, as verified in a direct measurement of the yield dependence on the FEL fluence (FIG. 2B). This quadratic dependence is significantly different from the rv9th order dependence observed in multiple vibrational excitations of H on Si(100) by STM.

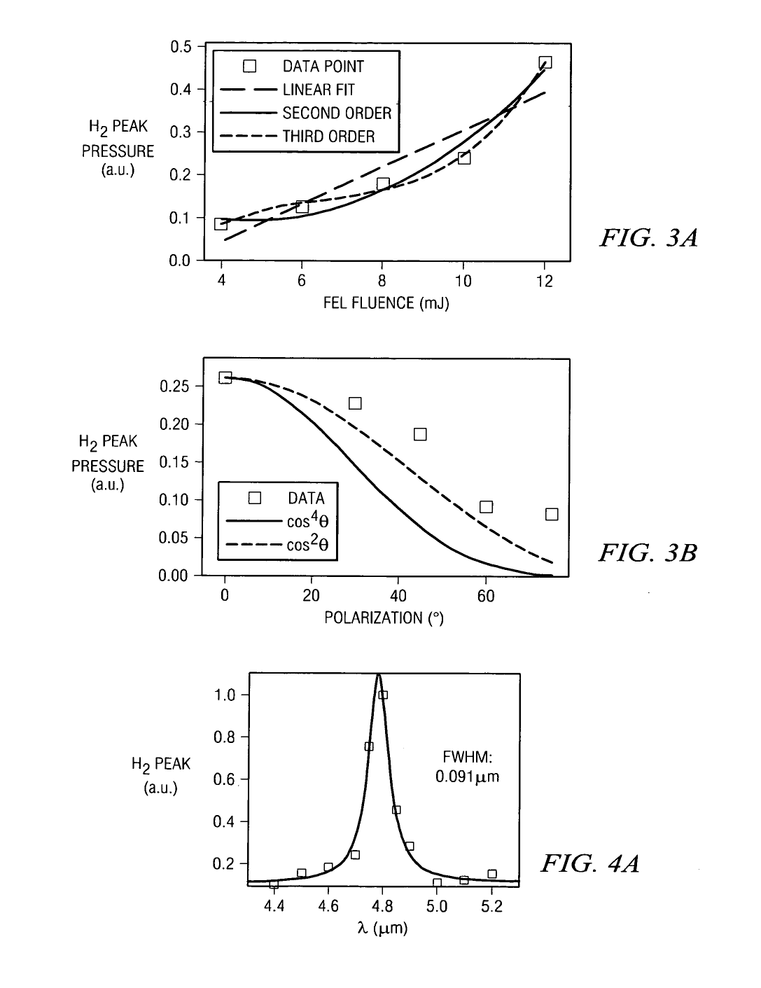

To further probe the nature of the desorption mechanism, the thermal desorption and IR induced desorption of a coadsorbed mixture of hydrogen and deuterium were also measured. H and D adatoms were coadsorbed and then the desorption yields of different species were measured (FIG. 3A). For thermal desorption, the ratio of the yields was H₂:HD:D₂=1:4.3:16.9. In the FEL-induced process, with the same HID coverage and the wavelength tuned to the Si—H stretch at 4.8 μm, the desorption ratio was found to be I:0.04:0.005, in stark contrast to the thermal data. Furthermore, the HD desorption yield was linear in photon flux (FIG. 3B) over the same range in which the H₂ yield was seen to be quadratic. These results support very different mechanisms for thermal desorption and IR induced desorption.

The invention employs a new technique of resonant photodesorption as a means of evolving a gas of interest from its storage materials for multiple purposes. For example, hydrogen atoms on the vast internal surface area of nanocrystal diamonds may be radiated by an infrared laser whose photon energy is resonant with the adsorbate-substrate vibrational stretch mode, leading to non-thermal, non-electronic desorption of hydrogen. Hydrogen gas is thus efficiently formed and released from this storage material for end uses, such as energy generation through chemical reaction with oxygen. This process of retrieving hydrogen takes place at room temperature and at near atmospheric pressures, avoiding the conventional problem of heat dissipation at extreme temperatures and pressures, thus enhancing the safety of the system. The proposed storage materials are transparent to infrared radiation, which not only allows infrared laser to effectively reach hydrogen atoms on the entire internal surface area, but also eliminates the adverse electronic excitation in the bulk of storage materials. The rate of evolving hydrogen can be easily controlled by varying the intensity of infrared radiation.

The method of the invention will have a colossal impact on the technology of retrieving a gas of interest, e.g., hydrogen, from its storage materials, thus reshaping the entire hydrogen economy and the energy structure of society.

The method of the invention has many applications. Thus, the facile desorption of hydrogen from adsorbate-substrates by the method of the invention enables the storage of hydrogen in a wider variety of substrates (e.g., carbon in all its forms, in particular, diamond) for subsequent desorption and use. Moreover, the desorption of hydrogen from the adsorbate-substrate, results in the formation of sites in the substrate that are reactive with a variety of functional radicals, e.g., free radicals of carbon, nitrogen, nitrogen oxides, hydrogen, chlorine oxides, hydrocarbons, polyaromatic hydrocarbons, halogens, cyanogens or sources of the radicals.

Thus, the method of the invention enables, e.g., Si epitaxy, Si nitridation, SiC formation, C[diamond] formation on Si, Si-catalytic substrate for hydrogenation or dehydrogenation reactions, use of laser quanta to establish zero heat of formation for an excited Si surface site, any C substrate/C surface site reactions to achieve low temp diamond growth and the like.

For example, graphitic carbon nanofibers (GCNFs) have edge-site carbon atoms terminated by hydrogen atoms, i.e. C—H bonds. The bulk hydrogen content of as-prepared GCNFs is ca. 0.29 wt %, and a very weak band for C—H bond stretching is evident in the IR spectrum of GCNFs. The method of the invention is applicable for the light-stimulated evolution of H₂ from as-prepared GCNF.

Hydrogenated Si surfaces absorb specific light quanta to evolve H₂ and generate an “excited” or “activated” Si surface site:

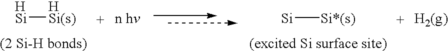

It was originally assumed that excited or reactive sites on the desorbed substrate would not “back add” hydrogen gas (H₂), although these reactive sites do react with two H atoms (radicals) to regenerate 2 Si—H surface bonds.

Reaction of these excited Si surface sites with single-radicals is very promising, since the kinetic barrier height for these reactions is near zero. All such additions should be exothermic. Some examples:

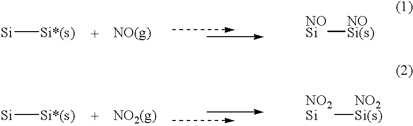

If NO and NO₂ additions occur on Si surfaces still containing some number of Si—H bonds, then further reaction of these surface-bound species will generate N₂ and H₂O, thereby acting as a “catalytic surface” for detoxifying the atmosphere of NOₓ pollutants. This application has “green-chemistry” or environmental applications. Such additions of nitrogen substituents to activated Si surface sites also provide low-temperature routes to surface nitridation of Si, a process of great application for electronic device applications.

Excited Si surface sites should likewise react with radical species in trace concentrations, such as polyaromatic hydrocarbon environmental pollutants (PAHs) known to oxidize under ambient conditions to form radicals and, therefore, serve as radical traps for these pollutants, another “green-chemistry” or environmental application.

Since single-radical species can be carcinogens, the trapping of trace-level radicals would find application in cancer prevention.

Reactions of excited Si surface sites with chemical reagents known to be facile sources of single-radical species might also occur rapidly. All such additions are also exothermic and should have very low kinetic barriers, since only very weak bonds need to be homolytically cleaved. Some examples:

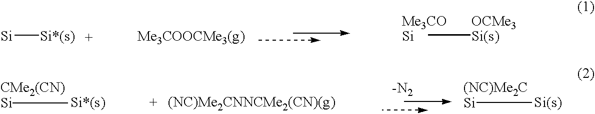

Reaction (1) is di-t-butyl peroxide addition to the excited Si surface. In reaction (2), azoisobutyronitrile (AIBN) adds two carbon radicals to the excited Si surface with loss of N₂. This latter reaction enables bonding a carbon radical onto the Si surface. If these carbon surface species subsequently add to alkenes or other unsaturated molecules, then a Si surface could be used to make C—C single bonds. This application relates to industrial hydrocarbon-building reactions using Si surfaces instead of noble metals to make C—C single bonds.

These excited surface sites should likewise react with simple diatomics like Cl₂ or Br₂ and even with cyanogen (CN)₂.

Knowing that the heat of formation of acetylene is +226.7 kJ/mol and taking the heat of formation of an excited Si surface site to be zero (produced by laser quanta), then the heat of reaction of the reaction shown below is exothermic by 430.7 kJ.

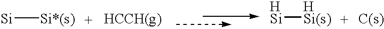

With a reaction exothermicity close to homolytic C—H bond dissociation energies, this reaction demonstrates H₂ extraction from a hydrocarbon. Thus, acetylene could be used as a source of H₂ for PEM fuel cells, etc.

Knowing that the heats of formation of 1,3-cyclohexadiene and benzene are +106.3 kJ/mol and +82.8 kJ/mol, respectively, and taking the heat of formation of an excited Si surface site to be zero (produced by laser quanta), then the heat of reaction of the reaction shown below is exothermic by 227.5 kJ.

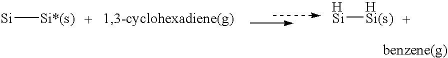

Although more exothermic than reaction (C) above, this reaction also might not occur rapidly if the mechanism reaction requires radical addition to excited Si surface sites. Homolytic cleavage of a C—H bond of this type requires ca. +360 kJ/mol of energy. Knowing that the heats of formation of ethane and ethylene are −84.7 kJ/mol and +52.3 kJ/mol, respectively, and taking the heat of formation of an excited Si surface site to be zero (produced by laser quanta), then the heat of reaction of the reaction shown below is exothermic by only 67 kJ.

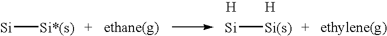

Extraction of H₂ from an alkane, such as ethane (C₂H₆), is much less exothermic than from a conjugated diene, as shown above. Homolytic cleavage of a C—H bond of the alkane type is ca. +418 kJ/mol.

It has now been unexpectedly determined that hydrogen gas does “back-add” to de-hydrogenated Si surfaces. This result is important and enables the performance of many other kinds of chemistry. Thus, excited surface sites remaining after desorption of hydrogen from substrates such as Si, C (e.g., nanodiamond) can extract hydrogen from hydrocarbons to re-hydrogenate the Si (or nanodiamond) surface, thus permitting a cyclical resonant molecular hydrogen dehydrogenation/surface re-hydrogenation process. In this fashion, only a small amount of Si or nanodiamond surface in the presence of laser light and a hydrocarbon “fuel” can produce hydrogen gas for a hydrogen/air fuel cell. Hydrocarbons can serve as room-temperature sources of clean hydrogen gas suitable for running a hydrogen/air fuel cell. The invention, therefore, solves many perplexing problems associated with the hydrogen storage problem for fuel cell technologies. These excited surface sites should likewise react with simple diatomics like Cl₂ or Br₂ and even with cyanogen (CN)₂.

An important aspect of using resonant light-initiated desorption of H₂ from Si or nanodiamond surfaces as efficient H₂ storage and delivery media is regeneration of the respective hydrogenated surfaces. Accomplishing this task corresponds to a refueling cycle. While treating hydrogen-depleted Si or nanodiamond material with atomic hydrogen or H₂ could regenerate the required hydrogenated surface, direct H₂ extraction from a liquid hydrocarbon would be a particularly consumer-friendly method for achieving on-board surface re-hydrogenation. In addition, resonate desorption of H₂ only at adsorbate-H (Si—H surface or nanodiamond C—H surface) frequencies followed by in situ surface-site extraction of H₂ from a hydrocarbon would provide a cyclical H₂ production/surface re-hydrogenation process requiring only a minimal effective surface area (and mass) of adsorbate such as Si or nanodiamond material.

H₂ extraction from hydrocarbons by methods other than reforming or partial oxidation is known. Direct elimination of H₂ from 1,4-cyclohexadiene forming benzene as a co-product is exothermic by 27 kJ/mol and occurs with an activation energy of only 183 kJ/mol.[Rico, R. J.; Page, M.; Doubleday, c., Jr. J. Am. Chem. Soc. 1992, 114, 1131.] This reaction is symmetry-allowed and has a low activation energy due to partial aromatization of the benzene ring in the transition state.

1,4-cyclohexadiene(g) [symbol] H₂(g)+benzene(g)

Noble metal surfaces reduce the activation energy of this process considerably. Both 1,4cyclohexadiene and 1,3-cyclohexadiene react with a Pt(111) surface at temperatures greater than 260 K to eliminate H₂ and form benzene [Su, X.; Shen, Y. R.; Somorjai, G. A. Chem. Phys. Leu. 1997, 280, 302.] This surface reaction occurs with an activation energy of only 58 kJ/mol. Similarly, octane adsorbed onto a Pt(100) 1×1 surface eliminates H₂ at temperatures below 200 K and undergoes exhaustive elimination of H₂ between 275-450 K along with formation of a surface-bound elemental carbon residue [Manner, W. L.; Girolami, G. S.; Nuzzo, R. G. Langmuir 1998, 14, 1716]. Although this reaction self-poisons the Pt surface, it demonstrates that surfaces can possess sufficient reactivity to extract much of the hydrogen content of octane near ambient temperature.

Certain Si surfaces imitate the dehydrogenation surface chemistry of Pt. 1-Methyl-1,4-cyclohexadiene absorbed onto a Si(111)7×7 surface undergoes dehydrogenation to form H₂ and toluene with an onset temperature of only 300 K. [MacPherson, C. D.; Hu, D. Q.; Doan, M.; Leung, K. T. Surf. Sci. 1994, 310, 231.] Thermal desorption profiles indicate maxima in toluene evolution at 350 K, 425 K, and at ca. 490 K. The mechanism for this process appears to be initial C—H bond cleavage to form Si—H surface groups.

A cyclical resonate H₂ desorption/surface re-hydrogenation process by H₂ extraction from hydrocarbons can be achieved with Si and nanodiamond materials. A mechanism is provided below for a Si substrate.

It is known that resonate desorption of H₂ from hydrogenated Si surfaces occurs at ambient temperature along with formation of excited Si surface sites, as shown below:

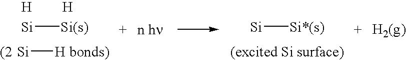

It is also known that re-hydrogenation of the Si surface occurs upon exposure to atomic hydrogen at low pressures or upon exposure to H₂ at pressures near 1 torr. Re-hydrogenation of the Si surface using H₂ gas occurs at a moderate rate (several minutes) at room temperature. Using a H—H bond energy of 432 kJ/mol and a Si—H bond energy of 318 kJ/mol, the heat of reaction for this surface re-hydrogenation reaction (shown below) is exothermic by 204 kJ:

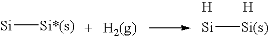

Assuming that the major contribution to the activation energy of this process is activation of H—H bond cleavage, then similar additions to the excited Si surface sites should occur at faster rates if the bond being cleaved has a bond energy less than 432 kJ/mol. Since allylic C(sp³)—H bond energies are ca. 360 kJ/mol and aliphatic C(sp³)—H bond energies are ca. 415 kJ/mol, both unsaturated and saturated hydrocarbons undergo adsorption onto these excited Si surface sites with C—H bond cleavage at rates faster than H₂ addition. Hydrocarbons having allylic C(sp³)-H bonds add to Si surface sites more readily than aliphatic hydrocarbons. The following reactions are exemplary:

(1) Reaction of resonantly dehydrogenated Si surfaces with 1,4-cyclohexadiene(g). Knowing that the heats of formation of 1,4-cyclohexadiene and benzene are +104.6 kJ/mol and +82.8 kJ/mol, respectively, and taking the heat of formation of an excited Si surface site to be zero (produced by laser quanta), then the heat of reaction of the reaction shown below is exothermic by 225.8 kJ.

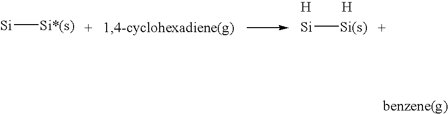

In addition, since the required C—H bond cleavage reactions of 1,4-cyclohexadiene involve allylic C—H bonds, the rate of this reaction at room temperature is reasonably high.

(2) Reaction of resonantly dehydrogenated Si surfaces with octane(g). Knowing that the heats of formation of octane and 1-octene are −208.6 kJ/mol and −82.9 kJ/mol, respectively, and taking the heat of formation of an excited Si surface site to be zero (produced by laser quanta), then the heat of reaction of the reaction shown below is exothermic by 78.3 kJ.

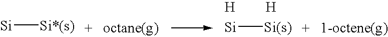

In addition, since the required C—H bond cleavage reactions of octane involve aliphatic CH bonds, the rate of this reaction at room temperature is slightly faster than that of H² addition to the Si excited surface.

(3) Irradiation of hydrogenated Si surfaces by Si—H resonate laser illumination in the presence of 1,4-cyclohexadiene or octane vapor. Continuous evolution of H₂ should be observed if a cyclical resonate H₂ desorption/surface re-hydrogenation process is established.

The invention thereby enables on-board vehicular hydrogen storage and delivery. This approach is based on the unprecedented and unexpected discovery [Z. Liu, L. C. Feldman, N. H. Tolk, Z. Zhang, P. I. Cohen, Science (2006) 1024-1026; J. Tully, Science (2006) 1004-1005] involving room temperature, non-thermal resonant photo-desorption of hydrogen from a Si(111) surface using tunable infrared radiation from a Free-Electron Laser (FEL). Hydrogen atoms on the extensive surface area of nano-crystal diamonds may be radiated by an infrared laser whose photon energy is resonant with the adsorbate-substrate (H—C) vibrational stretch mode, leading to direct non-thermal, non-electronic desorption of hydrogen.

The invention represents a realization of a long sought-after goal of controlling chemical reactions by selectively exciting a single vibrational mode. Hydrogen gas is thus efficiently formed and released from this storage material for end uses, such as fuel cell energy generation through chemical reaction with oxygen. This process of retrieving hydrogen takes place at room temperature (RT) and at near atmospheric pressures, avoiding the conventional problem of heat dissipation at extreme temperatures and pressures, thus enhancing the safety of the system. The proposed storage materials are transparent to infrared radiation, which not only allows infrared laser to effectively reach hydrogen adatoms on the entire internal surface area, but also eliminates the adverse electronic excitation in the bulk of storage materials. The rate of evolving hydrogen can be easily controlled by varying the intensity of infrared radiation.

Recharging the storage material with hydrogen can also be achieved at RT. Desorbing hydrogen from a surface results in the creation of highly chemically reactive surface sites that present opportunities for regeneration of hydrogenated surfaces by flowing H₂ gas or by flowing hydrocarbon vapors at standard pressures through the mass of diamond nano-crystals. While treating hydrogen-depleted Si or nanodiamond material with atomic hydrogen or H₂ regenerates the required hydrogenated surface, direct H₂ extraction from a liquid hydrocarbon is a particularly consumer-friendly method for achieving on-board surface re-hydrogenation.

Because of the high ratio of surface to bulk atoms, it is preferred, but not absolutely necessary, to use nano-crystalline diamond as the storage medium. The technology exists to scale up production of nanodiamonds on a large scale and low cost from materials available in abundance. The present global nanodiamond production far exceeds that of, for example, carbon nanotubes. Hydrogenated diamond surfaces are non-reactive, thermally stable, non-combustible, and non-toxic. It is further preferred to utilize presently available nanodiamond particles nominally 5 nm in diameter. Due to the light mass of carbon, the system will contain a high percentage of hydrogen. Preliminary calculations estimate hydrogen loading to be in the range of 2-9% by weight.

Based on the fact that bare surface diamond sites are highly reactive, the system may be loaded either by flowing H₂ gas or by flowing hydrocarbon vapors at standard pressures or lower through the mass of diamond nanocrystals. The hydrocarbons would suffer reduction as a result of coming into contact with the reactive sites, leaving the surface sites saturated with hydrogen. [A. V. Hamza, G. D. Kubiak and R H. Stulen, Surf. Sci. Lett., 206 (1988) L833]. In the absence of high temperatures or high pressures, it is a safe procedure. Hydrogen will not only be stored on the surfaces of the diamond nanoparticles, but also in the bulk, further improving the storage capacity.

Following the loading process, the nanodiamonds are placed in air-tight canisters for storage and transport in the vehicle.

At the appropriate time, an onboard shoe-box size laser can retrieve the hydrogen in the form of H₂ at room temperature (RT) which would then be delivered to hydrogen fuel cells.

The spent nanodiamond canisters are then returned for recharging with hydrogen or exchanged for a recharged canister. At room temperature, there is no significant degradation of the diamond surfaces in this process. Consequently, the nanodiamonds can be recycled indefinitely for this purpose. It is theorized that the method of the invention will make it feasible, and safe, to achieve on-board hydrogen storage that can power a car to travel more than 300 miles.

FIG. 1(a) shows the total desorption yield as a function of FEL wavelength. The desorption yield peaked at a wavelength of 4.8 μm, equivalent to 0.26 eV, the energy of the vibrational stretch mode of the Si—H bond at the terrace sites of the Si (111) surface. FIG. 1(b) shows the desorption yield as a function of FEL polarization with constant FEL fluence. The measured desorption yield follows the cos⁴ θ solid curve. These measurements, showing strong wavelength and polarization dependencies show that desorption is not caused by direct laser heating.

As a part of this study, we have examined the vibrational modes of C—H stretch bonds on various diamond films. In particular, we have measured FTIR absorbance in the range of 2500-3500 cm⁻¹ on one intrinsic CVD polycrystalline diamond film. Deconvolution of the spectrum shows four peaks within the well-known C—H stretching band (2780-3100 cm⁻¹) as shown in FIG. 2. From FIG. 2, it is clear that the four peaks arise from the vibrational modes of C—H stretching bonds on different diamond surfaces, with two peaks attributed to hydrogen on the diamond (111) surface and two to diamond (100) surface. This is supported by previous studies [E. Titus et al. Diamond & Related Materials 14 (2005)476-481; B. Dischler, C. Wild, W. Muller-Sebert and P. Koidl, Physica. B 185 (1993) 217-221]. Extending our desorption work on Si to diamond, we have performed laser induced desorption measurements on an intrinsic CVD polycrystalline diamond film. The preliminary measurements show a quadratic power dependence (FIG. 3(a)) and a less pronounced polarization dependence (FIG. 3(b)). The data suggests that the desorption is a multi-phonon process. Similar measurements on surfaces in which hydrogen and deuterium are co-adsorbed show that the process is not thermal in origin, i.e., is not due to local heating.

Our study demonstrates that tuned IR radiation can provide wavelength and polarization selective desorption in the H/Si (111) and H/diamond systems at room temperature. When implemented, this non-thermal desorption behavior avoids the conventional problem of heat dissipation at extreme temperatures and pressures, thus enhancing the safety of the system.

The nanodiamond storage material is transparent to IR radiation. This not only allows IR laser to effectively reach H adatoms on the entire surface area, but also eliminates the adverse electronic excitation in the bulk of storage materials, which also help prevent degrading of the storage material over multiple cycles of charging H. Once H adatoms are removed from the diamond surfaces, a density gradient of H atoms will form, pointing from the inside of the bulk to the surface. It will then drive the H atoms in the bulk to diffuse toward the surface and eventually desorb as H₂ molecules from the surface under the resonant IR excitation. Therefore, this new method is very efficient in retrieving H from the storage material without putting the system under high temperatures or high pressures.

H₂ extraction from hydrocarbons by methods other than reforming or partial oxidation is known. Direct elimination of H₂ from 1,4-cyclohexadiene forming benzene as a co-product (see below) is exothermic by 27 kJ/mol and occurs with an activation energy of only 183 kJ/mol.[Rico, R I.; Page, M.; Doubleday, c., Jr. J. Am. Chem. Soc. 1992, 114, 1131].

Noble metal surfaces reduce the activation energy of this process considerably. Both 1,4cyclohexadiene and 1,3-cyclohexadiene react with a Pt(111) surface at temperatures greater than 260 K to eliminate H₂ and form benzene [Su, X.; Shen, Y. R.; Somorjai, G. A. Chem. Phys. Lett. 1997, 280, 302]. Similarly, octane adsorbed onto a Pt(100) 1×1 surface eliminates H₂ at temperatures below 200 K and undergoes exhaustive elimination of H₂ between 275-450 K along with formation of a surface-bound elemental carbon residue [Manner, W. L.; Girolami, G. S.; Nuzzo, R G. Langmuir 1998, 14, 1716]. Certain Si surfaces imitate the dehydrogenation surface chemistry of Pt. 1-Methyl-1,4-cyclohexadiene absorbed onto a Si(111)7×7 surface undergoes dehydrogenation to form H₂ and toluene with an onset temperature of only 300 K.[MacPherson, C. D.; Hu, D. Q.; Doan, M.; Leung, K. T. Surf. Sci. 1994, 310, 231].

Re-hydrogenation of the Si surface occurs upon exposure to atomic hydrogen at low pressures or upon exposure to H₂ at pressures near 1 torr [M. Y. Mao, P. B. Miranda, D. S. Kim and Y. R Shen, Appl. Phys. Lett., 75, 3357 (1999)]. Re-hydrogenation using H₂ gas occurs at a moderate rate (several minutes) at room temperature. Assuming that the major contribution to the activation energy of this process is activation of H—H bond cleavage, then similar additions to the excited Si surface sites should occur at faster rates if the bond being cleaved has a bond energy less than 432 kJ/mol. Since allylic C(sp³)—H bond energies are ca. 360 kJ/mol and aliphatic C(sp³)—H bond energies are ca. 415 kJ/mol, both unsaturated and saturated hydrocarbons should undergo adsorption onto these excited Si surface sites with C—H bond cleavage at rates faster than H₂ addition. Hydrocarbons having allylic C(sp³)—H bonds should add to Si surface sites more readily than aliphatic hydrocarbons.

The present invention, therefore, provides a fundamentally new approach to hydrogen storage and extraction involving no extremes of temperature or pressure. This novel approach is based on room-temperature, non-thermal resonant photodesorption of hydrogen from adsorbate substrates such as silicon and diamond surfaces. Desorption results in a hydrogen-depleted surface, kinetically limited from substantial dimerization and chemically reactive, thus facilitating the recoating of the surfaces with hydrogen at room temperature (RT). These results offer new opportunities for storage, transport, and non-thermal extraction, all at normal temperatures and pressures.

The invention enables a compact, ambient temperature system of hydrogen storage/extraction employing infrared (IR) lasers and materials such as hydrogen saturated nano-scale diamond or porous silicon particulates with a large hydrogen/host weight ratio of 6 percent or more. The use of nanostructured materials makes possible H adsorption onto large surface area and also H diffusion into the bulk.

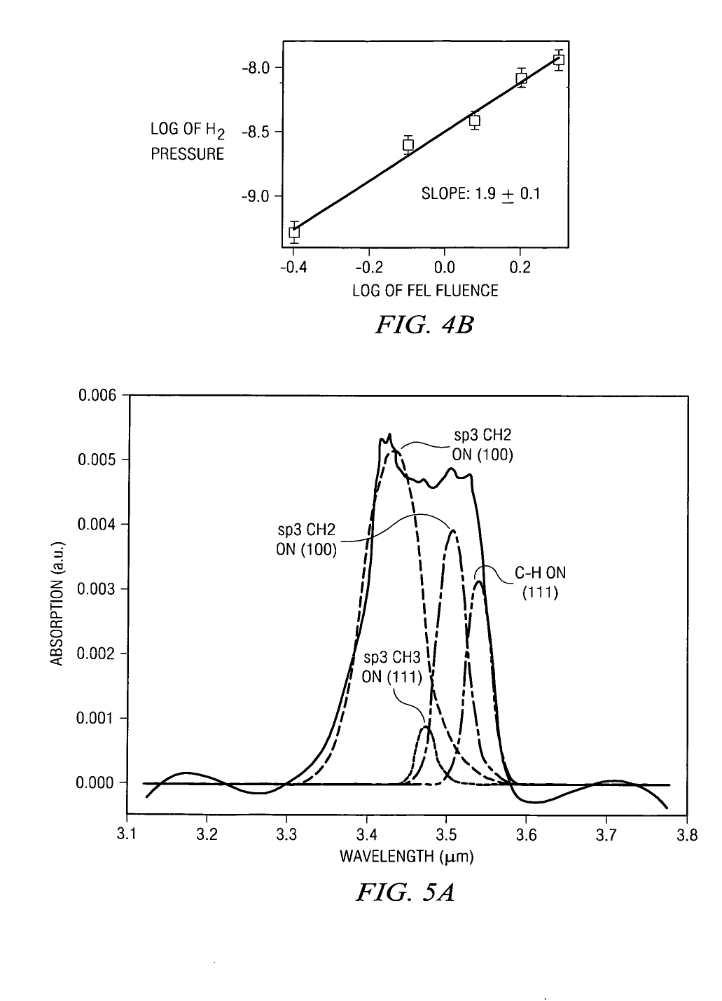

The data shown in FIG. 4 demonstrate that tuned near-IR light can provide wavelength selective desorption of hydrogen from a silicon substrate at RT with a direct relationship to laser intensity. We have also recorded strong polarization selective desorption of H₂ in the H/Si(111) system at RT.

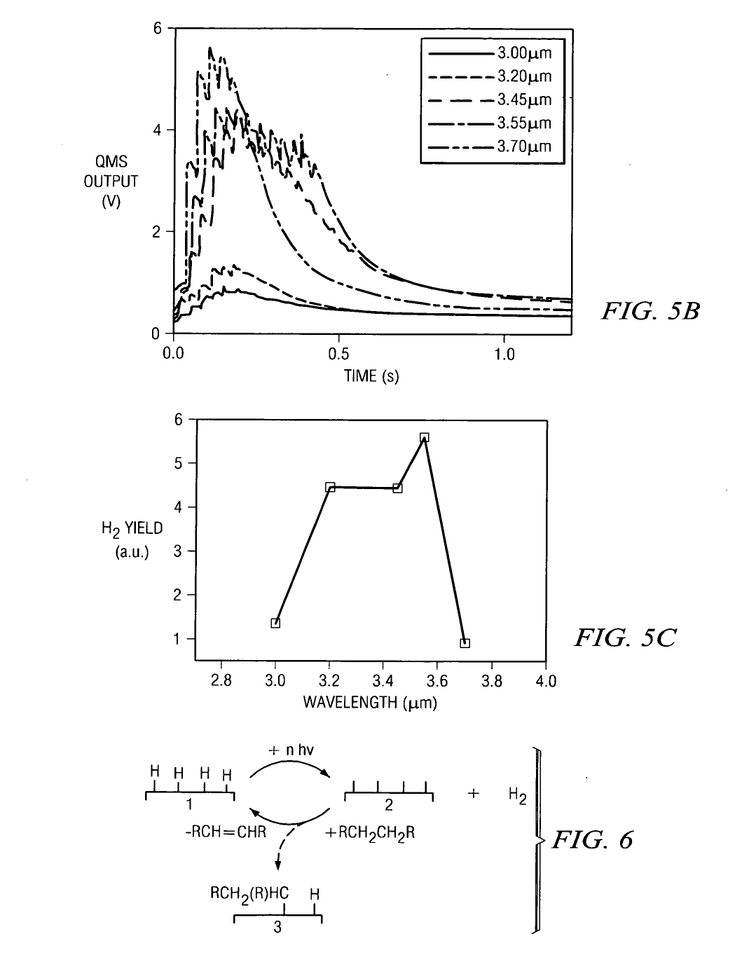

FIG. 5 shows initial data that indicates that the same effect occurs in the H/polycrystalline-diamond system. We have measured the vibrational modes of C—H stretch bonds on various polycrystalline diamond films. In FIG. 5, the left plot shows the FTIR absorbance measurements in the range of 2500-3500 cm⁻¹ performed on an intrinsic CVD diamond film. Deconvolution results in four peaks within the well-known C—H stretching bands (2780-3100 cm⁻¹). The other two plots (middle and right) clearly demonstrate the wavelength dependence of the hydrogen desorption on diamond polycrystalline surfaces. Note that in our studies on diamond, the wavelength dependence is much broader than in the case of silicon. The broadening is consistent with the fact that a layer of polycrystalline diamond exhibits a variety of faceted surfaces. Consequently, the broadening may be attributed primarily to the four peaks which arise from the absorption by the vibrational mode of the C—H bond on different diamond surfaces, with two peaks attributed to hydrogen on the diamond (111) surface and two to the diamond (100) surface.

This new demonstration of IR stimulated H₂ release from nanodiamond is particularly important in the context of this proposal because nanodiamond has been shown to contain more than 6% hydrogen by weight (with the possibility of greater H content [Prelas, M. A., Ghosh T. K., Loyalka, S. K., Tompson, R. V., Wide Bandgap Materials, 10 (2) 99-111 (2002)] and thus falls in the category of high hydrogen density materials, necessary for any hydrogen based system.

Nanodiamond derived from detonation diamond is a very attractive material for hydrogen storage. At 3-5 nm diameter particle size, nanodiamond has extensive surface area (>300 m²/g) and defect content (e.g. grain/twin boundaries) and is known [Reichart, 3D Microscopy of Hydrogen in Diamond and Proton Irradiated Graphite, CIMTEC 2006 (11 th International Conferences on Modem Materials and Technologies), Acireale, Sicily, Italy; (June 2006)] to readily incorporate hydrogen, and will be amenable to laser induced hydrogen release. A significant amount of nanodiamond is produced world-wide and manufacturing technology for megaton production exists. Interestingly, conventional weapon demilitarization can play a role in its production. Nanodiamond has been incorporated in bulk commercial plating operations in Russia for decades. Although still being characterized, the surface of nanodiamond is known [Shenderova, O. A., Zhirnov, V. V., and Brenner, D. W., Carbon nanostructures., Critical Rev. in Solid State and Materials Sciences, 27, 227 (2002)] to be chemically robust, being the subject of numerous activation and functionalization studies [Ultrananocrystalline Diamond: Synthesis, properties and Applications, edited by D. Gruen, A. Vul and O. Shenderova, NATO Science Series, Kluwer Acad. Publ. (2005); Surface Functionalization of Nanodiamond Particles via Atom Transfer Radical Polymerization, Lang Li, J. L. Davidson, and Charles M. Lukehart, Carbon, 2006, 44, 2308-2315]. Similarly, porous silicon nanostructures have been reported [V. Lysenko, F. Bidault, S. Aleeksev, J. Phys. Chem. B 109, 19711 (2005)] containing as much as 66 mmol/g of hydrogen corresponding to ˜6.6 wt. percent. The cited analysis clearly indicates that this silicon-based material is very competitive with all known hydrogen containing materials now being considered.

A light induced catalytic process is depicted in FIG. 6. Light-induced resonant desorption of H₂ from a hydrogenated Si surface, 1, at room temperature forms H₂ and a hydrogen-depleted surface of high free energy, 2. Exposure of this surface. to a generic hydrocarbon, RCH₂CH₂R, regenerates the hydrogenated surface by H₂ extraction and produces a hydrocarbon co-product having one additional degree of unsaturation. In the presence of excess saturated hydrocarbon, each cycle would produce one mole of H₂ and one mole of monounsaturated hydrocarbon coproduct for every mole of saturated hydrocarbon reacted. In the presence of near monolayer quantities of saturated hydrocarbon, continued H₂ extraction would produce successively higher degrees of hydrocarbon unsaturation.

### DEFINITIONS

The phrase functional radical is intended to mean an atom or molecular having one or more unpaired electron spins or a molecule that undergoes a chemical reaction to become or generate such a species, but not atomic or molecular hydrogen. The term program and/or the phrase computer program are intended to mean a sequence of instructions designed for execution on a computer system (e.g., a program and/or computer program, may include a subroutine, a function, a procedure, an object method, an object implementation, an executable application, an applet, a servlet, a source code, an object code, a shared library/dynamic load library and/or other sequence of instructions designed for execution on a computer or computer system). Group numbers corresponding to columns within the periodic table of the elements use the “New Notation” convention as seen in the CRC Handbook of Chemistry and Physics, 81^{st} Edition (2000).

The term substantially is intended to mean largely but not necessarily wholly that which is specified. The term approximately is intended to mean at least close to a given value (e.g., within 10% of). The term generally is intended to mean at least approaching a given state. The term coupled is intended to mean connected, although not necessarily directly, and not necessarily mechanically. The term proximate, as used herein, is intended to mean close, near adjacent and/or coincident; and includes spatial situations where specified functions and/or results (if any) can be carried out and/or achieved. The term distal, as used herein, is intended to mean far, away, spaced apart from and/or non-coincident, and includes spatial situation where specified functions and/or results (if any) can be carried out and/or achieved. The term deploying is intended to mean designing, building, shipping, installing and/or operating.

The terms first or one, and the phrases at least a first or at least one, are intended to mean the singular or the plural unless it is clear from the intrinsic text of this document that it is meant otherwise. The terms second or another, and the phrases at least a second or at least another, are intended to mean the singular or the plural unless it is clear from the intrinsic text of this document that it is meant otherwise. Unless expressly stated to the contrary in the intrinsic text of this document, the term or is intended to mean an inclusive or and not an exclusive or. Specifically, a condition A or B is satisfied by any one of the following: A is true (or present) and B is false (or not present), A is false (or not present) and B is true (or present), and both A and B are true (or present). The terms a and/or an are employed for grammatical style and merely for convenience.

The term plurality is intended to mean two or more than two. The term any is intended to mean all applicable members of a set or at least a subset of all applicable members of the set. The phrase any integer derivable therein is intended to mean an integer between the corresponding numbers recited in the specification. The phrase any range derivable therein is intended to mean any range within such corresponding numbers. The term means, when followed by the term “for” is intended to mean hardware, firmware and/or software for achieving a result. The term step, when followed by the term “for” is intended to mean a (sub)method, (sub)process and/or (sub)routine for achieving the recited result.

The terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. The terms “consisting” (consists, consisted) and/or “composing” (composes, composed) are intended to mean closed language that does not leave the recited method, apparatus or composition to the inclusion of procedures, structure(s) and/or ingredient(s) other than those recited except for ancillaries, adjuncts and/or impurities ordinarily associated therewith. The recital of the term “essentially” along with the term “consisting” (consists, consisted) and/or “composing” (composes, composed), is intended to mean modified close language that leaves the recited method, apparatus and/or composition open only for the inclusion of unspecified procedure(s), structure(s) and/or ingredient(s) which do not materially affect the basic novel characteristics of the recited method, apparatus and/or composition.

Unless otherwise defined, all technical and scientific terms used herein have the same meaning as commonly understood by one of ordinary skill in the art to which this invention belongs. In case of conflict, the present specification, including definitions, will control.

### CONCLUSION

The described embodiments and examples are illustrative only and not intended to be limiting. Although embodiments of the invention can be implemented separately, embodiments of the invention may be integrated into the system(s) with which they are associated. All the embodiments of the invention disclosed herein can be made and used without undue experimentation in light of the disclosure. Although the best mode of the invention contemplated by the inventor(s) is disclosed, embodiments of the invention are not limited thereto. Embodiments of the invention are not limited by theoretical statements (if any) recited herein. The individual steps of embodiments of the invention need not be performed in the disclosed manner, or combined in the disclosed sequences, but may be performed in any and all manner and/or combined in any and all sequences. Homologous replacements may be substituted for the substances described herein.

It can be appreciated by those of ordinary skill in the art to which embodiments of the invention pertain that various substitutions, modifications, additions and/or rearrangements of the features of embodiments of the invention may be made without deviating from the spirit and/or scope of the underlying inventive concept. All the disclosed elements and features of each disclosed embodiment can be combined with, or substituted for, the disclosed elements and features of every other disclosed embodiment except where such elements or features are mutually exclusive. The spirit and/or scope of the underlying inventive concept as defined by the appended claims and their equivalents cover all such substitutions, modifications, additions and/or rearrangements.

The appended claims are not to be interpreted as including means-plus-function limitations, unless such a limitation is explicitly recited in a given claim using the phrase(s) “means for” and/or “step for.” Subgeneric embodiments of the invention are delineated by the appended independent claims and their equivalents. Specific embodiments of the invention are differentiated by the appended dependent claims and their equivalents.

What is claimed is:

1. A method, comprising
forming reactive sites on an adsorbate-substrate by non-thermal, non-electronic resonant photodesorption of a gas from the adsorbate-substrate; and
reacting the reactive sites with a functional radical.

2. The method of claim 1, wherein the gas includes hydrogen and reacting includes re-hydrogenation of the reactive sites.

3. The method of claim 2, wherein the functional radical includes a hydrocarbon.

4. The method of claim 1, further comprising cyclically repeating the steps of forming and reacting.

5. The method of claim 1, wherein the adsorbate-substrate is selected from the group consisting of silicon, nanodiamond or nanocarbon.

6. The method of claim 1, wherein resonant photodesorption includes a vibrational stretch mode.

7. The method of claim 1, wherein the functional radical includes at least one member selected from the group consisting of NO or NO₂.

8. The method of claim 1, wherein the functional radical includes a polyaromatic hydrocarbon.

9. The method of claim 1, wherein the functional radical includes at least one member selected from the group consisting of Me₃COOCMe₃ or (NC)Me₂CNNCMe₂(CN).

10. The method of claim 1, wherein the functional radical includes at least one member selected from the group consisting of Cl₂, Br₂ or (CN)₂.

11. The method of claim 1, wherein the functional radical includes HCCH.

12. The method of claim 1, wherein the functional radical includes at least one member selected from the group consisting of 1,3-cyclohexadiene or 1,4-cyclohexadiene.

13. The method of claim 1, wherein the functional radical includes ethane.

14. The method of claim 1, wherein the functional radical includes octane.

15. A method, comprising
forming reactive sites on an adsorbate-substrate by non-thermal, non-electronic resonant photodesorption of a gas from the adsorbate-substrate;
reacting the reactive sites with a functional radical; and
cyclically repeating the steps of forming and reacting,
wherein the gas includes hydrogen and reacting includes re-hydrogenation of the reactive sites, the functional radical includes a hydrocarbon, the adsorbate-substrate is selected from the group consisting of silicon, nanodiamond or nanocarbon and resonant photodesorption includes a vibrational stretch mode.

16. A computer program, comprising computer or machine readable program elements translatable for implementing the method of claim 1.
